(19) United States
(12) Patent Application Publication
(10) Pub. No.: US 2020/0066406 A1
(43) Pub. Date: Feb. 27, 2020

(54) TOOL FOR IDENTIFYING OCCURRENCE OF ACUTE INCIDENT SYMPTOMATIC OF MENTAL CONDITION OR DISORDER

(71) Applicants: Centre for Addiction and Mental Health, Toronto (CA); Memotext Corporation, Toronto (CA)

(72) Inventors: Sean Andrew Kidd, Toronto (CA); Amos Adler, Toronto (CA); Hesamaldin Nekouei, Richmond Hill (CA)

(21) Appl. No.: 16/109,394

(22) Filed: Aug. 22, 2018

(51) Int. Cl. G16H 50/20; G16H 80/00; A61B 5/16; A61B 5/00; A61B 5/11

(52) U.S. Cl. CPC G16H 50/20; G16H 80/00; A61B 5/165; A61B 5/746; A61B 5/747; A61B 2562/0219; A61B 5/0022; A61B 5/6898; A61B 2560/0242; A61B 2562/0204; A61B 5/1123

(57) ABSTRACT

A tool is described for objectively identifying and providing assessment of acute psychotic episodes, such as auditory hallucinations, symptomatic of a mental condition or disorder. The tool can include an audio interface on a microphone-equipped networked mobile wireless telecommunication computing device which a patient can use to determine whether the perceived sounds are real or hallucinatory. If the microphone fails to detect ambient sounds indicating that the sounds are real, the application can provide a remote alert signal so that appropriate parties (e.g. the patient's treatment team or emergency medical personnel) can take action.

### TECHNICAL FIELD

The present technology relates to a tool for identification of occurrences of acute instances of a key symptom of psychotic illness, namely auditory hallucinations.

### BACKGROUND

Psychosis broadly and auditory hallucinations specifically are present in several major mental illnesses, including bipolar disorder, post-traumatic stress disorder (PTSD), and most notably schizophrenia spectrum illnesses. Auditory hallucinations involve hearing voices and other sounds when such sounds are not objectively present.

One objective in treating schizophrenia and other illnesses involving psychosis is to provide medication which can obviate the symptoms and allow those suffering with the condition to live in the community. However, because of the complexity of psychosis and the fact that psychiatry remains an inexact science, medications are not always completely effective and can, for a substantial number of sufferers, only partially treat distressing auditory hallucinations or be entirely ineffective in that area.

If a medication regimen is not effective, or if a patient is non-adherent to the regimen, or if titration or medication adjustment is required, symptoms such as hallucinations may remain present, and may impede community functioning and quality of life for the patient. At a minimum, this is information that should be brought to the attention of the person(s) providing treatment, and the occurrence of acute auditory hallucinatory episodes may also indicate a serious worsening of the condition that places the patient and/or others in the community at risk. However, the nature of psychosis makes it very difficult for a patient to “self diagnose” auditory hallucinations.

### SUMMARY

According to the present disclosure, a tool is described for objectively identifying and providing assessment of acute psychotic episodes, such as auditory hallucinations, symptomatic of a mental condition or disorder. The tool can include an audio interface on a microphone-equipped networked mobile wireless telecommunication computing device which a patient can use to determine whether those sounds are real or hallucinatory. If the microphone fails to detect ambient sounds indicating that the sounds are real, the application can provide a remote alert signal so that appropriate parties (e.g. the patient's treatment team) are notified and can take action.

According to one aspect of the disclosure, a method for providing a remote alert signal identifying potential occurrence of an acute auditory hallucinatory episode is described. The method comprises monitoring, by at least one processor of a first networked mobile wireless telecommunication computing device, for a deliberate overt activation action by a user, wherein the activation action represents an indication that the user is hearing sounds. Responsive to the activation action, the processor(s) will use at least one microphone on the first networked mobile wireless telecommunication computing device to monitor ambient sounds. The processor(s) will test the ambient sounds against a threshold, and responsive to at least one instance for which the processor(s) determine, in response to the activation action, that the ambient sounds fail to satisfy the threshold, the processor(s) will transmitting at least one alert signal, via a network to which the first networked mobile wireless telecommunication computing device is coupled, to at least one remote receiving device beyond the first networked mobile wireless telecommunication computing device.

In some embodiments, the processor(s) will test the ambient sounds against the threshold locally on the first networked mobile wireless telecommunication computing device. In some embodiments, the processor(s) will test the ambient sounds against the threshold remotely by transmitting the ambient sounds from the first networked mobile wireless telecommunication computing device to a remote computer system and receiving threshold testing results from the remote computer system at the first networked mobile wireless telecommunication computing device.

In some embodiments, the processor(s) will transmit the alert signal(s) only after a predetermined number of instances within a predetermined time period for which the processor(s) determine in response to the activation action, that the ambient sounds fail to satisfy the threshold.

In some embodiments, the alert signal(s) may be one or more of a text message, a pager message, a telephone call and an e-mail message, and the remote receiving device(s) may comprise one or more additional networked mobile wireless telecommunication computing device(s) associated with a medical professional involved in treatment of the user.

The threshold may be, for example, a minimum volume threshold, or a minimum confidence level associated with natural language processing of the ambient sounds.

In another aspect, the present disclosure is directed to a networked mobile wireless telecommunication computing device comprising at least one processor, at least one microphone coupled to the at least one processor, at least one input device coupled to the at least one processor, at least one communications interface coupled to the at least one processor, wherein the at least one communications interface is coupled to at least one wireless communication module, and at least one memory coupled to the at least one processor, the memory containing instructions which, when executed by the at least one processor, cause the at least one processor to implement the above-described method for providing a remote alert signal identifying potential occurrence of an acute auditory hallucinatory episode.

In yet another aspect, the present disclosure is directed to a tangible computer-readable medium containing computer-usable instructions for execution by at least one processor of a networked mobile wireless telecommunication computing device, wherein the instructions, when executed by the at least one processor, cause the at least one processor to implement the above-described method for providing a remote alert signal identifying potential occurrence of an acute auditory hallucinatory episode.

### BRIEF DESCRIPTION OF THE DRAWINGS

These and other features of the invention will become more apparent from the following description in which reference is made to the appended drawings wherein:

FIG. 1 shows in schematic form an illustrative system for providing a remote alert signal identifying potential occurrence of an acute auditory hallucinatory episode;

FIG. 2 is a flow chart showing an illustrative method for providing a remote alert signal identifying potential occurrence of an acute auditory hallucinatory episode;

FIG. 3 shows an illustrative structure for an illustrative function for capturing the amplitude of audio;

FIG. 4 shows an illustrative structure for an illustrative function for building a sine waveform based on detected amplitude;

FIG. 5 shows an illustrative structure for an illustrative function for applying detected sound to a waveform;

FIG. 6 shows an illustrative computer system in respect of which aspects of the present disclosure may be implemented; and

FIG. 7 shows an illustrative networked mobile wireless telecommunication computing device in respect of which aspects of the present disclosure may be implemented.

### DETAILED DESCRIPTION

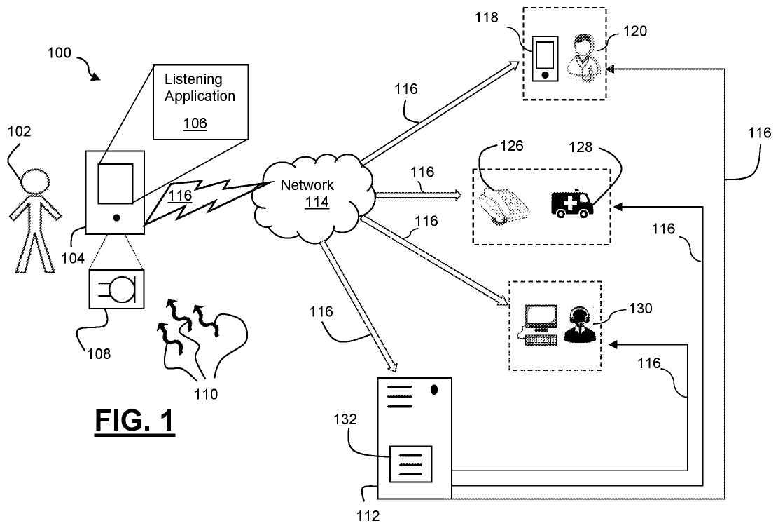

Reference is now made to FIG. 1, which shows in schematic form an illustrative system, indicated generally by reference 100, for providing a remote alert signal identifying potential occurrence of an acute auditory hallucinatory episode.

A first networked mobile wireless telecommunication computing device, represented for simplicity of illustration by smartphone 104, is associated with a user 102 who has been diagnosed with psychosis. The smartphone 104 may be owned by the user 102, or merely possessed by the user 104 under a loan, lease, bailment or other suitable arrangement. The smartphone 104 is merely one representative example of a networked mobile wireless telecommunication computing device, which may also be a tablet, smartwatch or other suitable device possessing a microphone, suitable wireless communication hardware and sufficient processing capacity. The wireless communication hardware may operate in conjunction with other communication hardware, for example a WiFi signal from a smartwatch or tablet may communicate with a router having a wired connection to one or more network.

The processor(s) of the smartphone 104 execute a listening application 106, which monitors for a deliberate overt activation action by the user 102. Importantly and critically, the activation action represents an affirmative, unambiguous indication by the user that the user 102 is hearing voices or other sounds. For example, the listening application 106 may have a virtual button on a screen thereof that says “I'm hearing things” or “I am hearing voices” or “Are the voices real?” or “Discretely check the background for noises”, or something similar. Alternatively, the listening application 106 may have an activation action that involves a specific sequence of button pushes, or a specific gesture, such as vigorously shaking the smartphone 104 in a manner that can be unambiguously be detected by an onboard accelerometer. The listening application 106 may run in the background for rapid access, or may be launched when needed. In the latter case, the act of launching the listening application 106 may represent an affirmative, unambiguous indication by the user that the user 102 is hearing sounds. The listening application 106 may be a stand-alone application, or may be a component of a larger software application providing additional features and functionality, for example to assist an individual with psychosis with living in the community.

In response to the activation action by the user 102, the processor(s) executing the listening application 106 on the smartphone 104 uses at least one microphone 108 on the smartphone 104 to monitor ambient sounds, shown as arrows 110. In some embodiments, the microphone 108 may be inactive prior to the activation action, so that only ambient sounds 110 after the activation action are monitored. In other embodiments, the processor(s) executing the listening application 106 may cause the microphone 108 to remain active in the background. For example, the processor(s) executing the listening application 106 may continuously record ambient sounds 110 and store a predetermined duration (e.g. a preceding 5 seconds, 10 seconds, etc.) thereof in a rolling buffer so that ambient sounds 110 immediately prior to the activation action may be used, either alone or in addition to ambient sounds 110 following the activation action.

Optionally, the listening application 106 may display a waveform or other representation of the ambient sounds 110 on a screen of the smartphone 104.

The processor(s) executing the listening application 106 tests the ambient sounds 110 against a threshold to determine whether the user 102 is experiencing an acute auditory hallucinatory episode. The threshold is designed to test whether there is evidence in the ambient sounds 110 to support the indication by the user 102 that the user 102 is hearing voices or other sounds, e.g. whether it can be inferred that the voices/sounds heard by the user 102 are actually present in the ambient sounds 110, or may be an auditory hallucination. Depending on the desired bias in terms of Type I error (false positive) vs. Type II error (false negative), various thresholds can be used, alone or in combination. For example, the threshold may be a minimum volume threshold, or may be a minimum confidence level associated with natural language processing of the ambient sounds 110, e.g. whether or not a natural language processing engine can identify spoken works in the ambient sounds 110. These are merely some representative examples of thresholds, and are not intended to be limiting.

The processor(s) executing the listening application 106 may test the ambient sounds 110 against the threshold locally on the smartphone 104, or remotely by transmitting the ambient sounds 110 from the networked mobile wireless telecommunication computing device to a remote computer system 112 through one or more networks 114 (e.g. comprising one or more wireless networks, intranets, cellular networks, the publically switched telephone network (PSTN) and/or the Internet) to which the smartphone 104 is coupled and receiving threshold testing results from the remote computer system 112 at the smartphone 104. In the latter case, the remote computer system 112 may have far superior processing capacity to the smartphone 104 so as to more rapidly execute the required processing, e.g. natural language processing.

Optionally, if the processor(s) executing the listening application 106 determine that the ambient sounds 110 satisfy the threshold, indicating that the microphone 108 has detected ambient sounds 110 supporting an inference that the sounds heard by the user 102 are actually present, the processor(s) executing the listening application 106 may provide a visual and/or audible notification to the user 102. This may provide reassurance to the user 102 that the user 102 is hearing actual ambient sounds and is not experiencing an auditory hallucination.

If the processor(s) executing the listening application 106 determine that the ambient sounds 110 fail to satisfy the threshold, this indicates that the ambient sounds 110 detected by the microphone 108 do not support an inference that the sounds heard by the user 102 are actually present, and therefore that the sounds may be an auditory hallucination. Accordingly, responsive to the processor(s) executing the listening application 106 determining that the ambient sounds fail to satisfy the threshold, the processor(s) executing the listening application 106 will cause the smartphone 104 to wirelessly transmit one or more alert signals 116 that identify the user 102 and indicate that the user 102 may be experiencing an auditory hallucination. Optionally, the processor(s) executing the listening application 106 may also provide a visual and/or audible notification to the user 102. The alert signal(s) 116 are sent, via the network(s) 114, to at least one remote receiving device beyond the smartphone 104. Examples of remote receiving devices include at least one second networked mobile wireless telecommunication computing device 118 associated with a medical professional 120 involved in treatment of the user 102, a telephone or dispatch system 126 associated with an ambulance or paramedic service 128, and a dedicated monitoring center 130. The alert signal(s) 116 can be one or more of a text message, a pager message, a telephone call, an e-mail message, a push notification or other types of signal. The alert signal(s) 116 may indicate that the user 102 may be experiencing an auditory hallucination either explicitly, or implicitly (e.g. a push notification on a dedicated application running on a smartphone or other device associated with a medical professional 120 involved in treatment of the user 102).

The processor(s) may cause transmission of the alert signal 106 in response to a single instance for which the processor(s) determines, in response to the activation action, that the ambient sounds fail to satisfy the threshold. In other embodiments, the alert signal(s) 116 will only be generated after a predetermined number of instances within a predetermined time period for which, following an activation action by the user 102, the processor(s) executing the listening application 106 determine that the ambient sounds 110 fail to satisfy the threshold. Additionally, in some embodiments, the number of activation actions by the user, and the number of times that the ambient sounds 110 fail to satisfy the threshold, may be recorded and transmitted to inform clinicians of patient wellness between appointments.

As noted above, the smartphone 102 is merely one representative example of a networked mobile wireless telecommunication computing device. Where the device (e.g. smartphone 104) has telephone connectivity through the network(s) 114, the alert signal 116 may be, for example, an automated telephone call, text message, pager message or e-mail message sent according to conventional protocols. Alternatively, the alert signal 116 may be transmitted through the network(s) 114 to another system, e.g. remote computer system 112, for further processing. For example, profile information 132 about the user 102 may be stored on the remote computer system 112, and the remote computer system 112 can use the profile information 132 to embellish the alert signal 116. For example, the alert signal 116 may consist of a unique identifier for the user 102, or a limited data set (e.g. a unique identifier and timestamp and/or location). The remote computer system 112 can forward the embellished alert signal 116, which can then be forwarded to, for example, one or more of a device 118 associated with a medical professional 120 involved in treatment of the user 102, a telephone or dispatch system 126 associated with an ambulance or paramedic service 128, and a dedicated monitoring center 130. Alternatively or additionally, the remote computer system 112 may update an electronic medical record of the user based on the alert signal 116. The alert signal 116 may trigger an alert within the electronic medical record and/or an alarm on a web portal.

Optionally, where available, the alert signal 116 can include location information (e.g. from a location processor of the smartphone 104). For example, if a profile of the user 102 indicates that he or she may pose a danger to himself/herself or others in the event of auditory hallucinations, the alert signal 116 can be used to dispatch emergency medical personnel 128 to the location of the smartphone 104, which is expected to be at (or at least near) the location of the user 102. In such cases, the alert signal can also provide additional information, such as one or more photographs of the user 102 to assist emergency medical personnel 128 in identifying the user 102 when they arrive.

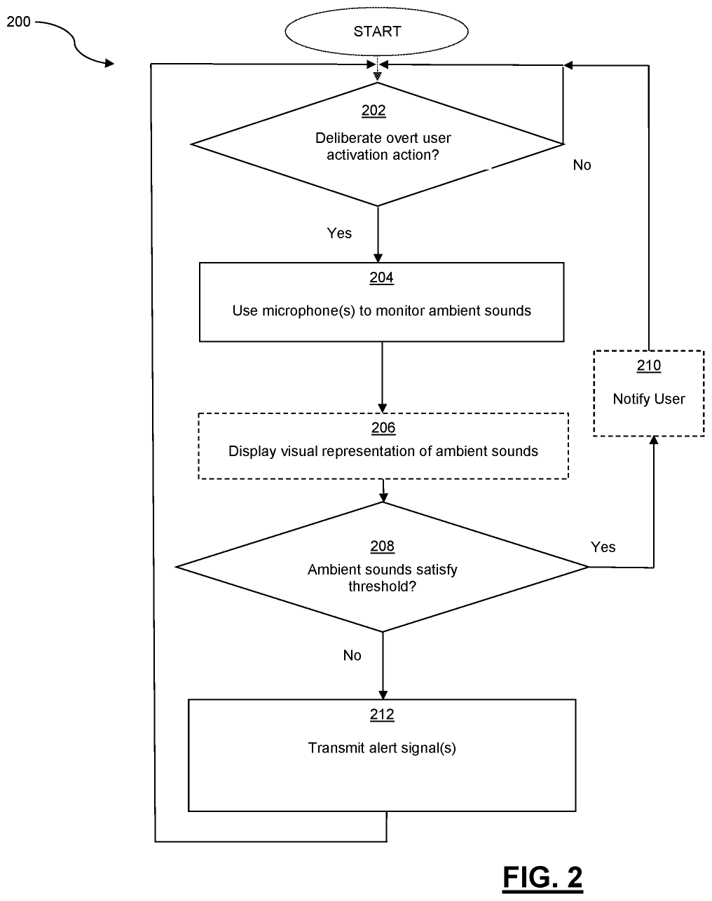

Reference is now made to FIG. 2, in which an illustrative method for providing a remote alert signal identifying potential occurrence of an acute auditory hallucinatory episode is indicated generally at reference 200.

At step 202, the method 200 monitors, by at least one processor of a first networked mobile wireless telecommunication computing device, for a deliberate overt activation action by a user. As noted above, the activation action, when detected, represents an indication that the user is hearing sounds. If the activation action is detected (a “yes” at step 202), the method 200 proceeds to step 204; otherwise (a “no” at step 202) the method 200 continues to monitor at step 202.

At step 204, responsive to the activation action being detected, the processor(s) using at least one microphone on the first networked mobile wireless telecommunication computing device to monitor ambient sounds. In one illustrative implementation, the Cordova-Plugin-Media sound detector, available from Apache for both Android and iOS platforms at https://cordova.apache.org/docs/en/latest/reference/cordova-plugin-media/, may be used to access the microphone. This package allows the microphone to capture any ambient sounds around the networked mobile wireless telecommunication computing device, and to play, pause and stop recorded audio, change the volume and read the current position of playing audio. In one illustrative embodiment, ambient sounds are captured by the interval function (shown below) every 0.4 seconds. The amplitude range is 0 to 1, with voice capture sensitivity set to anything more than 0.06 of the amplitude rate to eliminate very low volume noises. This is merely one illustrative implementation and is not limiting.

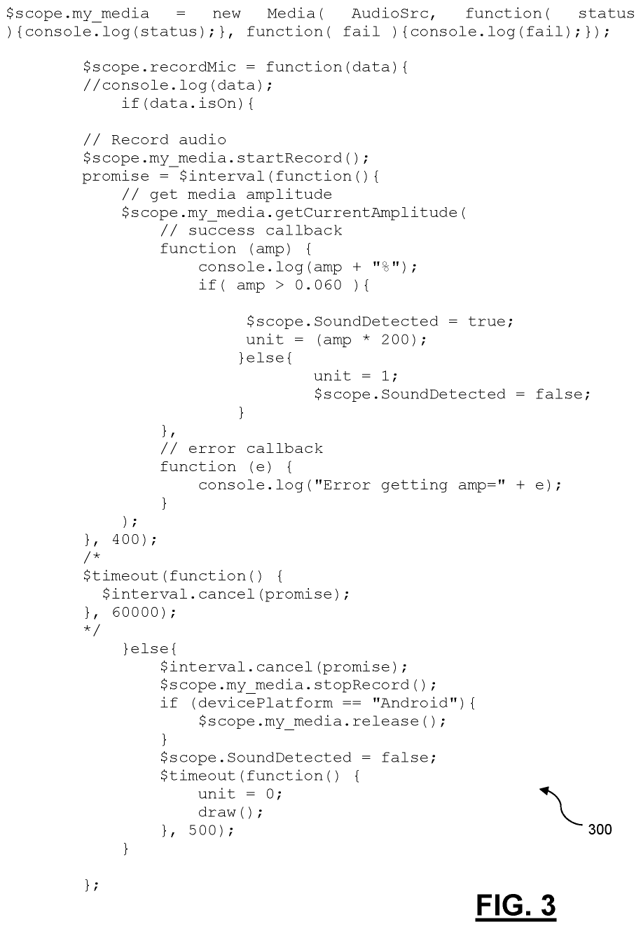

The function for capturing the amplitude of audio in the Cordova-Plugin-Media is: media.getCurrentAmplitude(mediaSuccess, [mediaError]);. The structure shown at reference 300 in FIG. 3 is used to implement this function.

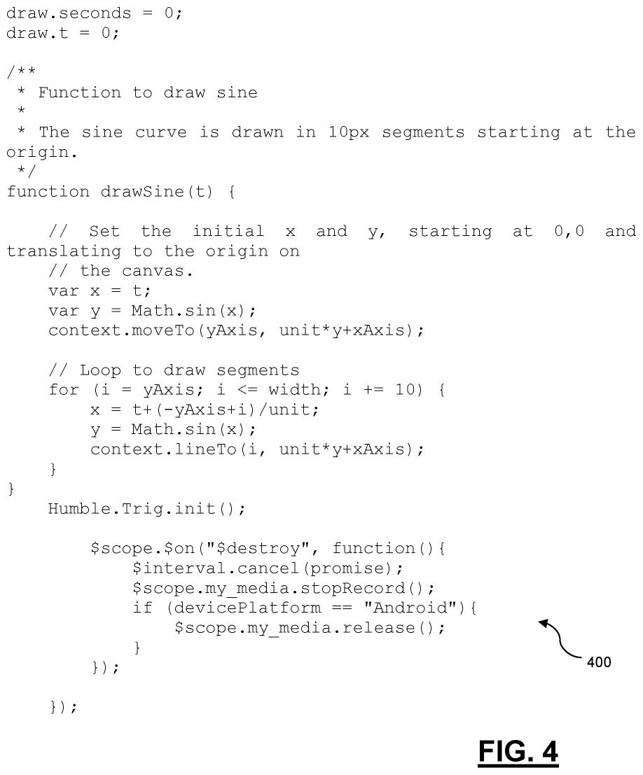

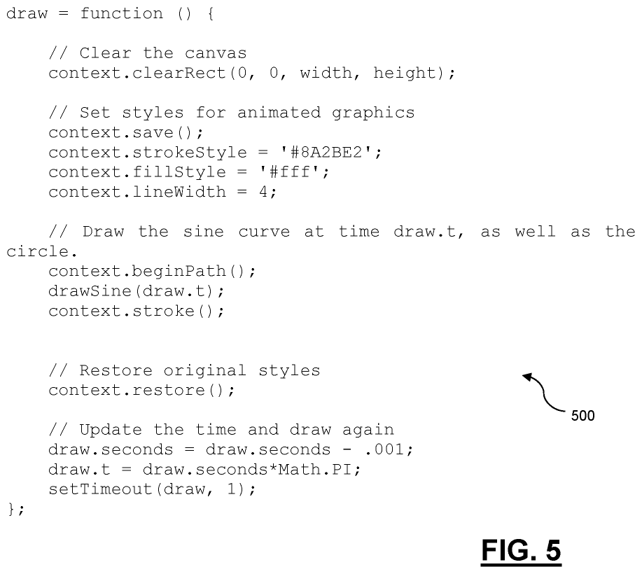

Returning to FIG. 2, after step 204 the method 200 proceeds to optional step 206, where the processor(s) may display a visual representation of the ambient sounds on a display of the first networked mobile wireless telecommunication computing device. In one illustrative implementation, the ambient sounds are visualized as a sine waveform (other visual representations may also be used). A first function, shown at 400 in FIG. 4, may be used to build the sine waveform based on detected amplitude. The amplitude is magnified to enable identification of minor changes in the wave form. The sine curve is drawn in 10 px segments starting at the origin in this function. The height of the sine waveform is changing based on detected sound amplitude with a parameter called “unit”. This allows the waveform to be plotted on a display of the first networked mobile wireless telecommunication computing device. The detected sound may then be applied to the waveform using the function shown at 500 in FIG. 5, according to the following recursive steps:

1. Clear the screen in position (x, y) with context.clearRect;

2. Save cleared screen;

3. Define color and width of waveform;

4. Draw sine curve at moment of t;

5. Update moment of t; and

6. Return to step (1).

After optional step 206, or from step 204 where optional step 206 is omitted, the method 200 proceeds to step 208, where the processor(s) test the ambient sounds against a threshold. As noted above, this may be done locally or remotely, and the threshold may be, for example, a minimum volume threshold, a minimum confidence level associated with natural language processing of the ambient sounds, or another suitable threshold.

If the processor(s) determine at step 208 that the ambient sounds satisfy the threshold (a “yes” at step 208), this indicates that the ambient sounds detected by the microphone supporting an inference that the sounds heard by the user are actually present, and the method proceeds to optional step 210 to provide a visual and/or audible notification to the user, and then returns to step 202.

If the processor(s) determine at step 208 that the ambient sounds fail to satisfy the threshold (a “no” at step 208), this indicates that the ambient sounds detected by the microphone(s) do not support an inference that the sounds heard by the user are actually present, and therefore that the sounds may be an auditory hallucination. Accordingly, responsive to the processor(s) determining that the ambient sounds fail to satisfy the threshold (a “no” at step 208), the method 200 proceeds to step 212 where the processor(s) transmit an alert signal, via a network to which the first networked mobile wireless telecommunication computing device is coupled, to at least one remote receiving device beyond the first networked mobile wireless telecommunication computing device. The alert signal may be transmitted, for example, in the manner described above. After step 212, the method 200 returns to step 202, or may optionally end.

Although illustrative embodiments have been described with respect to individuals who have been diagnosed with psychosis, it will be appreciated that this is merely by way of illustrative example. The present disclosure is not limited to psychosis, and may be applied in respect of any psychiatric disorder for which auditory hallucinations are a symptom.

As can be seen from the above description, the technology described herein represents significantly more than merely using categories to organize, store and transmit information and organizing information through mathematical correlations. The technology is in fact an improvement to the technology of monitoring previously diagnosed psychiatric conditions. The technology described herein provides for objective external assessment of whether a user-identified experience is an actual auditory sensory experience or is an occurrence of an acute auditory hallucinatory episode, and for notification of relevant third parties where a hallucinatory episode is detected. This facilitates the ability of relevant personnel to timely respond to the incident to prevent harm. As such, the technology is confined to psychiatric monitoring applications. Moreover, it is to be appreciated that the present technology is not directed to methods of medical treatment or even to methods of diagnosing a particular disorder; it is applied, inter alia, where a diagnosis has already been made by a human medical practitioner. The technology provides an objective technique for detection of potential acute incidents within the context of an existing diagnosis, eliminating subjectivity by either doctor or patient. In this sense, the present technology provides a manually activated mechanical diagnostic tool to replace subjective perception with objective measurement. In this sense, the present technology, while innovative in its application and implementation, is analogous in its result to a manually activated heart monitor for a patient already diagnosed with a cardiac condition. Just as a heart monitor replaces a subjective assessment of “my heart is racing” with an objective measure of actual heart rate that can be relied upon by user and practitioner, the present technology replaces an inherently subjective and unreliable comparison between perceived and actual sounds with a reliable objective assessment in real time.

The present technology may be embodied within a system, a method, a computer program product or any combination thereof. The computer program product may include a computer readable storage medium or media having computer readable program instructions thereon for causing a processor to carry out aspects of the present technology. The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing.

A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present technology may be assembler instructions, instruction-set-architecture (ISA) instructions, machine instructions, machine dependent instructions, microcode, firmware instructions, state-setting data, or either source code or object code written in any combination of one or more programming languages, including an object oriented programming language or a conventional procedural programming language. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to implement aspects of the present technology.

Aspects of the present technology have been described above with reference to flowchart illustrations and/or block diagrams of methods, apparatus (systems) and computer program products according to various embodiments. In this regard, the flowchart and block diagrams in the Figures illustrate the architecture, functionality, and operation of possible implementations of systems, methods and computer program products according to various embodiments of the present technology. For instance, each block in the flowchart or block diagrams may represent a module, segment, or portion of instructions, which comprises one or more executable instructions for implementing the specified logical function(s). It should also be noted that, in some alternative implementations, the functions noted in the block may occur out of the order noted in the Figures. For example, two blocks shown in succession may, in fact, be executed substantially concurrently, or the blocks may sometimes be executed in the reverse order, depending upon the functionality involved. Some specific examples of the foregoing may have been noted above but any such noted examples are not necessarily the only such examples. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

It also will be understood that each block of the flowchart illustrations and/or block diagrams, and combinations of blocks in the flowchart illustrations and/or block diagrams, can be implemented by computer program instructions. These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer readable program instructions may also be stored in a computer readable storage medium that can direct a computer, other programmable data processing apparatus, or other devices to function in a particular manner, such that the instructions stored in the computer readable storage medium produce an article of manufacture including instructions which implement aspects of the functions/acts specified in the flowchart and/or block diagram block or blocks. The computer readable program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

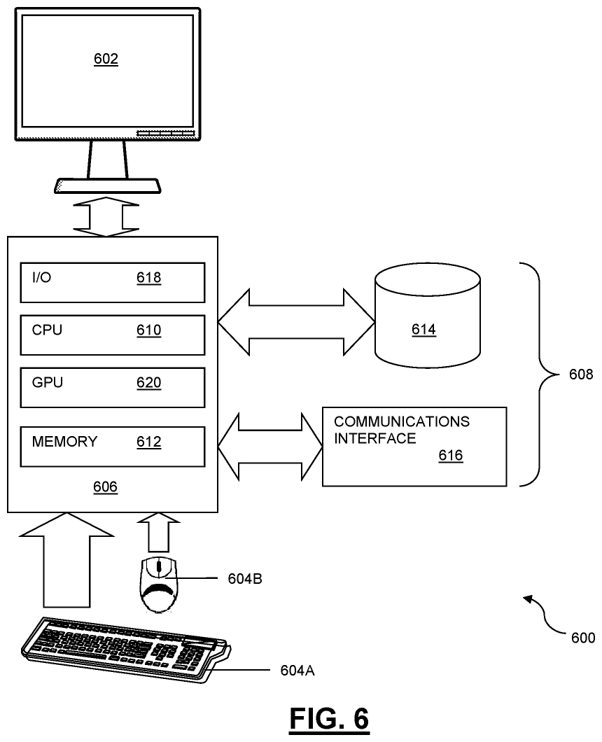

An illustrative computer system in respect of which aspects of the technology herein described may be implemented is presented as a block diagram in FIG. 6. For example, the illustrative computer system 600 may be used to implement the remote computer system 112, as part of a dispatch system 126 associated with an ambulance or paramedic service 128, and/or part of a dedicated monitoring center 130, all as shown in FIG. 1.

The illustrative computer system is denoted generally by reference numeral 600 and includes a display 602, input devices in the form of keyboard 604A and pointing device 604B, computer 606 and external devices 608. While pointing device 604B is depicted as a mouse, it will be appreciated that other types of pointing device, or a touch screen, may also be used.

The computer 606 may contain one or more processors or microprocessors, such as a central processing unit (CPU) 610. The CPU 610 performs arithmetic calculations and control functions to execute software stored in an internal memory 612, preferably random access memory (RAM) and/or read only memory (ROM), and possibly additional memory 614. The additional memory 614 may include, for example, mass memory storage, hard disk drives, optical disk drives (including CD and DVD drives), magnetic disk drives, magnetic tape drives (including LTO, DLT, DAT and DCC), flash drives, program cartridges and cartridge interfaces such as those found in video game devices, removable memory chips such as EPROM or PROM, emerging storage media, such as holographic storage, or similar storage media as known in the art. This additional memory 614 may be physically internal to the computer 606, or external as shown in FIG. 6, or both.

The computer system 600 may also include other similar means for allowing computer programs or other instructions to be loaded. Such means can include, for example, a communications interface 616 which allows software and data to be transferred between the computer system 600 and external systems and networks. Examples of communications interface 616 can include a modem, a network interface such as an Ethernet card, a wireless communication interface, or a serial or parallel communications port. Software and data transferred via communications interface 616 are in the form of signals which can be electronic, acoustic, electromagnetic, optical or other signals capable of being received by communications interface 616. Multiple interfaces, of course, can be provided on a single computer system 600.

Input and output to and from the computer 606 is administered by the input/output (I/O) interface 618. This I/O interface 618 administers control of the display 602, keyboard 604A, external devices 608 and other such components of the computer system 600. The computer 606 also includes a graphical processing unit (GPU) 620. The latter may also be used for computational purposes as an adjunct to, or instead of, the (CPU) 610, for mathematical calculations.

The various components of the computer system 600 are coupled to one another either directly or by coupling to suitable buses.

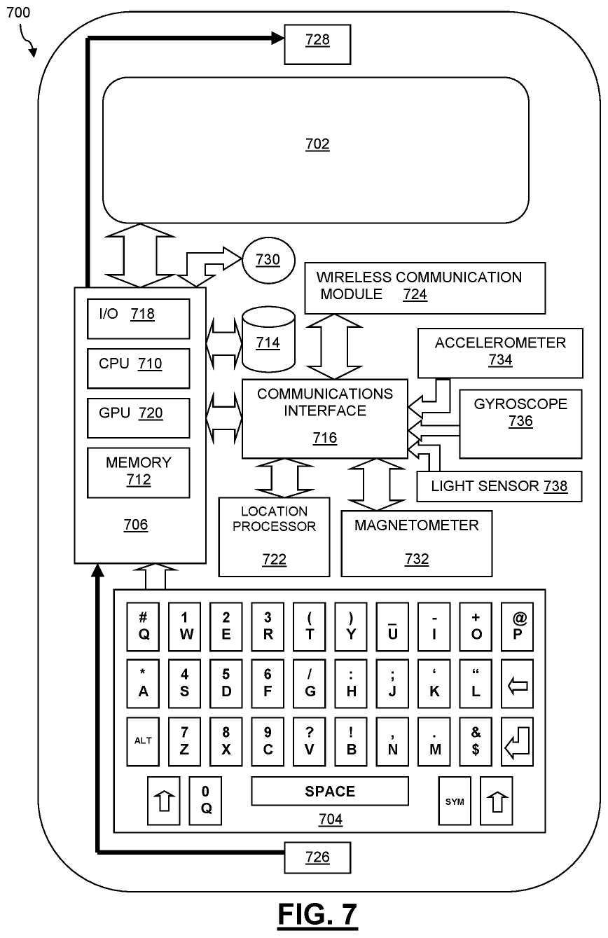

FIG. 7 shows an illustrative networked mobile wireless telecommunication computing device in the form of a smartphone 700. Thus, the smartphone 700 is an illustrative representation of the networked mobile wireless telecommunication computing device shown as a smartphone 104 in FIG. 1.

The smartphone 700 includes a display 702, an input device in the form of keyboard 704 and an onboard computer system 706. The display 702 may be a touchscreen display and thereby serve as an additional input device, or as an alternative to the keyboard 704. The onboard computer system 706 comprises a central processing unit (CPU) 710 having one or more processors or microprocessors for performing arithmetic calculations and control functions to execute software stored in an internal memory 712, preferably random access memory (RAM) and/or read only memory (ROM) is coupled to additional memory 714 which will typically comprise flash memory, which may be integrated into the smartphone 700 or may comprise a removable flash card, or both. The smartphone 700 also includes a communications interface 716 which allows software and data to be transferred between the smartphone 700 and external systems and networks. The communications interface 716 is coupled to one or more wireless communication modules 724, which will typically comprise a wireless radio for connecting to one or more of a cellular network, a wireless digital network or a Wi-Fi network. The communications interface 716 will also typically enable a wired connection of the smartphone 700 to an external computer system. A microphone 726 and speaker 728 are coupled to the onboard computer system 706 to support the telephone functions managed by the onboard computer system 706. Of note, the microphone 726 may be used to detect ambient sounds (e.g. ambient sounds 110 as shown in FIG. 1). A location services module 722 (e.g. including GPS receiver hardware) may also be coupled to the communications interface 716 to support navigation operations by the onboard computer system 706. One or more cameras 730 (e.g. front-facing and/or rear facing cameras) may also be coupled to the onboard computer system 706. A magnetometer 732 may also be coupled to the communications interface 716 to support navigation operations by the onboard computer system 706; the magnetometer functions as an electronic compass and gathers data used to determine the direction of magnetic North. An accelerometer 734 and gyroscope 736 are coupled to the communications interface 716 to gather data about movement of the smartphone 700. A light sensor 738 is also coupled to the communications interface 716. Input and output to and from the onboard computer system 706 is administered by the input/output (I/O) interface 718, which administers control of the display 702, keyboard 704, microphone 726, speaker 728 and camera(s) 730. The onboard computer system 706 may also include a separate graphical processing unit (GPU) 720. The various components are coupled to one another either directly or by coupling to suitable buses.

Without limitation, any one or more of the display 702 (if a touchscreen), keyboard 704, microphone 726, camera 730, accelerometer 734 and gyroscope 736 and light sensor 738 may be considered an input device that can be used to monitor for a deliberate overt activation action by the user.

The term “computer system”, “data processing system” and related terms, as used herein, is not limited to any particular type of computer system and encompasses servers, desktop computers, laptop computers, networked mobile wireless telecommunication computing devices such as smartphones, tablet computers, as well as other types of computer systems.

Thus, computer readable program code for implementing aspects of the technology described herein may be contained or stored in the memory 712 of the onboard computer system 706 of the smartphone 700 or the memory 612 of the computer 606, or on a computer usable or computer readable medium external to the onboard computer system 706 of the smartphone 700 or the computer 606, or on any combination thereof.

Finally, the terminology used herein is for the purpose of describing particular embodiments only and is not intended to be limiting. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description has been presented for purposes of illustration and description, but is not intended to be exhaustive or limited to the form disclosed. Many modifications and variations will be apparent to those of ordinary skill in the art without departing from the scope of the claims. The embodiment was chosen and described in order to best explain the principles of the technology and the practical application, and to enable others of ordinary skill in the art to understand the technology for various embodiments with various modifications as are suited to the particular use contemplated.

Certain illustrative embodiments have been described by way of example. It will be apparent to persons skilled in the art that a number of variations and modifications can be made without departing from the scope of the invention as defined in the claims. In construing the claims, it is to be understood that the use of a computer, and in particular a networked mobile wireless telecommunication computing device, to implement the embodiments described herein is essential.

What is claimed is:

1. A method for assessing an acute psychotic episode symptomatic of a mental condition or disorder and providing a remote alert signal indicating the acute auditory hallucinatory episode, the method comprising:
monitoring, by at least one processor of a first networked mobile wireless telecommunication computing device, for a deliberate overt activation action by a user, wherein the activation action represents an indication that the user is hearing sounds;
responsive to the activation action, the at least one processor using at least one microphone on the first networked mobile wireless telecommunication computing device to monitor ambient sounds;
the at least one processor testing the ambient sounds against a threshold; and
responsive to at least one instance for which the at least one processor determines, in response to the activation action, that the ambient sounds fail to satisfy the threshold, the at least one processor transmitting at least one alert signal, via a network to which the first networked mobile wireless telecommunication computing device is coupled, to at least one remote receiving device beyond the first networked mobile wireless telecommunication computing device.

2. The method of claim 1, wherein the at least one processor testing the ambient sounds against the threshold comprises testing the ambient sounds against the threshold locally on the first networked mobile wireless telecommunication computing device.

3. The method of claim 1, wherein the at least one processor testing the ambient sounds against the threshold comprises testing the ambient sounds against the threshold remotely by transmitting the ambient sounds from the first networked mobile wireless telecommunication computing device to a remote computer system and receiving threshold testing results from the remote computer system at the first networked mobile wireless telecommunication computing device.

4. The method of claim 1, wherein the at least one processor transmits the at least one alert signal only after a predetermined number of instances within a predetermined time period for which the at least one processor determines, in response to the activation action, that the ambient sounds fail to satisfy the threshold.

5. The method of claim 1, wherein:
the alert signal is at least one of a text message, a pager message, a telephone call and an e-mail message; and
the at least one remote receiving device comprises at least one second networked mobile wireless telecommunication computing device associated with a medical professional involved in treatment of the user.

6. The method of claim 1, wherein the threshold is a minimum volume threshold.

7. The method of claim 1, wherein the threshold is a minimum confidence level associated with natural language processing of the ambient sounds.

8. A networked mobile wireless telecommunication computing device, comprising:
at least one processor;
at least one microphone coupled to the at least one processor;
at least one input device coupled to the at least one processor;
at least one communications interface coupled to the at least one processor, wherein the at least one communications interface is coupled to at least one wireless communication module;
at least one memory coupled to the at least one processor, the memory containing instructions which, when executed by the at least one processor, cause the at least one processor to implement a method for assessing an acute psychotic episode symptomatic of a mental condition or disorder and providing a remote alert signal indicating the acute auditory hallucinatory episode by:
monitoring, by the at least one processor, of the at least one input device for a deliberate overt activation action by a user, wherein the activation action represents an indication that the user is hearing sounds;
responsive to the activation action, the at least one processor using the at least one microphone to monitor ambient sounds;
the at least one processor testing the ambient sounds against a threshold; and
responsive to at least one instance for which the at least one processor determines, in response to the activation action, that the ambient sounds fail to satisfy the threshold, the at least one processor transmitting at least one alert signal, via a network to which the first networked mobile wireless telecommunication computing device is coupled via the at least one communications and the at least one wireless communication module, to at least one remote receiving device beyond the networked mobile wireless telecommunication computing device.

9. The networked mobile wireless telecommunication computing device of claim 8, wherein the at least one processor testing the ambient sounds against the threshold comprises testing the ambient sounds against the threshold locally on the networked mobile wireless telecommunication computing device.

10. The networked mobile wireless telecommunication computing device of claim 8, wherein the at least one processor testing the ambient sounds against the threshold comprises testing the ambient sounds against the threshold remotely by transmitting the ambient sounds from the networked mobile wireless telecommunication computing device to a remote computer system and receiving threshold testing results from the remote computer system at the networked mobile wireless telecommunication computing device.

11. The networked mobile wireless telecommunication computing device of claim 8, wherein the at least one processor transmits the at least one alert signal only after a predetermined number of instances within a predetermined time period for which the at least one processor determines, in response to the activation action, that the ambient sounds fail to satisfy the threshold.

12. The networked mobile wireless telecommunication computing device of claim 8, wherein:
the alert signal is at least one of a text message, a pager message, a telephone call and an e-mail message; and
the at least one remote receiving device comprises at least one other networked mobile wireless telecommunication computing device associated with a medical professional involved in treatment of the user.

13. The networked mobile wireless telecommunication computing device of claim 8, wherein the threshold is a minimum volume threshold.

14. The networked mobile wireless telecommunication computing device of claim 8, wherein the threshold is a minimum confidence level associated with natural language processing of the ambient sounds.

15. A tangible computer-readable medium containing computer-usable instructions for execution by at least one processor of a networked mobile wireless telecommunication computing device, wherein the instructions, when executed by the at least one processor, cause the at least one processor to implement a method for assessing an acute psychotic episode symptomatic of a mental condition or disorder and providing a remote alert signal indicating the acute auditory hallucinatory episode by:
monitoring, by the at least one processor, for a deliberate overt activation action by a user, wherein the activation action represents an indication that the user is hearing sounds;
responsive to the activation action, the at least one processor using at least one microphone on the first networked mobile wireless telecommunication computing device to monitor ambient sounds;
the at least one processor testing the ambient sounds against a threshold; and
responsive to at least one instance for which the at least one processor determines, in response to the activation action, that the ambient sounds fail to satisfy the threshold, the at least one processor transmitting at least one alert signal, via a network to which the first networked mobile wireless telecommunication computing device is coupled, to at least one remote receiving device beyond the networked mobile wireless telecommunication computing device.

16. The computer-readable medium of claim 15, wherein the instructions cause the at least one processor to test the ambient sounds against the threshold by testing the ambient sounds against the threshold locally on the networked mobile wireless telecommunication computing device.

17. The computer-readable medium of claim 15, wherein the instructions cause the at least one processor to test the ambient sounds against the threshold by transmitting the ambient sounds from the networked mobile wireless telecommunication computing device to a remote computer system and receiving threshold testing results from the remote computer system at the networked mobile wireless telecommunication computing device.

18. The computer-readable medium of claim 15, wherein:
the alert signal is at least one of a text message, a pager message, a telephone call and an e-mail message; and
the at least one remote receiving device comprises at least one other networked mobile wireless telecommunication computing device associated with a medical professional involved in treatment of the user.

19. The computer-readable medium of claim 15, wherein the threshold is a minimum volume threshold.

20. The computer-readable medium of claim 15, wherein the threshold is a minimum confidence level associated with natural language processing of the ambient sounds.

21. The computer-readable medium of claim 15, wherein the instructions, when executed by the at least one processor, cause the at least one processor to transmit the at least one alert signal only after a predetermined number of instances within a predetermined time period for which the at least one processor determines, in response to the activation action, that the ambient sounds fail to satisfy the threshold.
